(19) United States
(12) Patent Application Publication
(10) Pub. No.: US 2014/0107555 A1
(43) Pub. Date: Apr. 17, 2014

(54) ANTIMICROBIAL MULTILAYER WOUND DRESSING

(71) Applicant: Covidien LP, Mansfield, MA (US)

(72) Inventor: Harish Patel, Norfolk, MA (US)

(73) Assignee: Covidien LP, Mansfield, MA (US)

(21) Appl. No.: 14/136,710

(22) Filed: Dec. 20, 2013

Related U.S. Application Data
(63) Continuation-in-part of application No. 13/426,134, filed on Mar. 21, 2012, now abandoned.
Application No. 13/426,134 is a continuation of application No. 11/716,008, filed on Mar. 9, 2007, now abandoned.
Application No. 11/716,008 is a continuation-in-part of application No. 13/330,092, filed on Dec. 19, 2011.
Application No. 13/330,092 is a continuation of application No. 10/278,072, filed on Oct. 23, 2002, now Pat. No. 8,100,872.
(60) Provisional application No. 60/790,813, filed on Apr. 11, 2006.
(60) Provisional application No. 60/790,814, filed on Apr. 11, 2006.

(51) Int. Cl. A61L 26/00

(52) U.S. Cl. CPC A61L 26/0066
USPC 602/48; 424/445; 514/635; 424/642; 424/604

(57) ABSTRACT

A wound dressing includes one or more layers containing an antimicrobial agent and optionally at least one of: a chelating agent, a second antimicrobial agent, a zinc-containing agent, a cell-signaling agent, and an additional active ingredient or agent.

### BACKGROUND

1. Field

The present disclosure relates to multilayer wound dressings containing an antimicrobial agent, a sponge formed from such a dressing, and methods of fabricating such dressings and sponge.

2. Related Art

One common technique for reducing the cost of providing health care services is to reduce the amount of time of in-patient hospital stays, and to reduce to a minimum the amount of person-to-person interaction between a patient and healthcare professionals. A related problem is that of the incidence of bacterial infection of post operative and other wounds. Such infections not only increase the demands on the resources of our healthcare system through lengthened hospital stays, but also require treatment with antibiotics. Due to the misuse of antibiotics, finding an effective treatment for such infections can prove difficult. Moreover, tremendous resources are needed to constantly develop new antibiotics to replace other such drugs that have been rendered ineffective.

In the area of wound care, a variety of wound dressings have been suggested. However, such wound dressings possess various deficiencies and shortcomings.

For example, a number of wound dressings have been proposed which include various antimicrobial agents. U.S. Pat. No. 6,369,289 discloses a cellulosic dressing material having a calculated amount of PHMB applied thereto.

U.S. Pat. No. 4,655,756 discloses an article comprising a non-woven material treated with PHMB at a concentration ranging from 500 to 5000 ppm.

U.S. Pat. No. 5,098,417 relates to a wound dressing constructed for the controlled release of an active agent into the wound.

Abstracted Chinese patent publication number CN1170564 A discloses a wound dressing comprising a zinc-calcium alginate in the form of a nonwoven fabric.

U.S. Pat. No. 5,931,800 discloses a wound dressing including zinc and/or various alginates.

U.S. Pat. No. 5,238,685 discloses a wound dressing comprising a mixed salt alginate, possibly in the form of calcium alginate fibers, and an antimicrobial agent.

U.S. Pat. No. 5,759,570 discloses a multilayer wound dressing wherein the wound contacting layer thereof comprises a bioabsorbable and hydrophilic polymeric material.

U.S. Pat. No. 6,599,525 discloses a dressing having a first skin-facing surface, and a discontinuous coating of a semi-solid composition having an ointment-like feel overlying a portion of the first surface.

U.S. Pat. No. 4,699,792 discloses a self-adhesive medicinal plaster comprising a plurality of active ingredient elements spaced from each other and disposed on a carrier web.

U.S. Pat. No. 4,643,180 discloses a surgical dressing having an adhesive, and wherein PHMB is provided in the adhesive at a concentration on the order of 1 to 20% by weight.

U.S. Patent Application Publication No. 2002/0022660 discloses a deep-penetrating antimicrobial composition comprising antimicrobial components and a combination of surfactants that do not include anionic surfactants.

U.S. Patent Application Publication No. 2004/0047763 discloses an antimicrobial water-based system formulated to disinfect a catheter, etc., which includes approximately 10 to 200 mg of tetrasodium EDTA for each milliliter of water contained in the system.

U.S. Patent Application Publication No. 2004/0028722 discloses a wound dressing comprising a microbial-derived cellulose dressing material containing PHMB at a concentration on the order of 2700-7900 ppm.

U.S. Patent Application Publication No. 2004/0142019 discloses a microbial-derived cellulose wound dressing provided in the form of a hydrogel which may also contain PHMB and other additives.

U.S. Patent Application Publication No. 2005/0019380 discloses a wound dressing formed from a microbial-derived cellulose capable of donating liquid to a dry substrate, as well as absorbing exudate from a wound. The dressing may be treated to also contain PHMB.

U.S. Pat. No. 3,797,494 discloses a bandage for use in the continuous administration of drugs to the skin or mucosa which includes a reservoir that can comprise a distinct layer containing a plurality of microcapsules and a drug release rate controlling microporous membrane material which meters the flow of drug transfer to the skin.

U.S. Pat. No. 3,731,683 describes the bandage for the topical administration of therapeutically effective quantities of a topically active substance.

U.S. Pat. No. 3,598,122 discloses a bandage for the continuous administration of a systematically active drug via absorption through the skin or oral mucosa comprising a backing member and a reservoir having a wall distant from the backing member, the wall being permeable so as to permit passage of the drug in a controlled manner for absorption through the skin.

U.S. Patent Application Publication No. 2005/0048139 discloses compositions which may include an antimicrobial agent as well as a zinc-containing compound, which reportedly serves to prevent irritation of the skin.

U.S. Pat. No. 4,211,227, discloses a nonwoven surgical sponge material comprising a layered fabric having an inner core or a substantially hydrophilic material disposed adjacent at least one outer or surface layer, or between a pair of outer layers, of a substantially hydrophobic material.

U.S. Pat. No. 5,707,736 discloses a dry, disposable, polymeric product having sustained-release antimicrobial activity formed from a polymeric material having an amine salt antimicrobial agent incorporated therein.

U.S. Pat. No. 4,643,181 also describes a surgical dressing and a process for making a surgical dressing.

U.S. Pat. No. 4,675,347 relates to an antimicrobial latex composition and a method of manufacturing a shaped article by repeatedly dipping an article into the composition and drying.

U.S. Pat. No. 4,678,704 describes an impregnated fabric material comprising a fabric substrate to which has been bonded an active cationic impregnant along with an anionic indicator dye in combination with a further cationic component, wherein the dye bonds to the further cationic component more readily than to the substrate and the further cationic component competes with the impregnant for bonding to the dye. The cationic impregnant may be a polymeric biguanide, e.g., PHMB.

U.S. Pat. No. 4,837,079 relates to a method for making an antimicrobially active, non-woven web comprising the steps of forming an unbounded fibrous web, applying throughout the unbonded fibrous web an uncured binder and PHMB hydrochloride as an antimicrobial agent.

U.S. Pat. No. 5,141,803 relates to a moistened wipe for cleaning and delivering a cationic biocide comprising a flexible absorbent nonwoven substrate impregnated with an aqueous composition comprising defined amounts of potassium sorbate, citric acid, disodium ethylenediaminetetraacetate, a cationic biocide selected from a defined group which includes PHMB hydrochloride and the remainder water.

U.S. Pat. No. 5,156,843 relates to a composition of matter comprising a material provided with interstices having solid particles residing therein. The material is a member selected from the group consisting of joined fibers, woven fabric, non-woven fabric, paper, woven cloth, non-woven cloth, foamed plastic and sponge. The solid particles are from about one to about 100 microns in diameter and contain a substantially continuous network of pores open to the exterior of the particles, with a functional substance retained in the pores. The functional substance may be a biocidal substance that can prevent or retard bacterial, microbial, germ or fungal growth.

U.S. Pat. No. 5,498,416 relates to a process for protection of prostheses, implants and/or catheters, of temporary or permanent implantable materials against bacterial colonization and infection.

U.S. Pat. No. 5,700,742 relates to a method of treating a textile material to inhibit microbial growth which comprises applying to the textile material an oligo- or polymeric biguanide or salt thereof with an inorganic acid or an organic acid having a pK value above 4.5 followed by a strong organic acid having a pK value below 4.5 and free from any aliphatic or oxyalkylene chain containing 12 or more carbon atoms.

U.S. Pat. No. 5,856,248 relates to cellulose fibers and products comprising cellulose fibers treated to absorb body secretions while substantially decreasing microbial growth, the fibers being chemically modified in a two-stage process comprising a first stage treatment with a water soluble salt of a transition metal and an alkali and a second stage treatment with a solution of a bisbiguanide compound, thereby forming a bond between the cellulose fibers, the transition metal and the bisbiguanide compound.

U.S. Pat. No. 5,869,073 relates to a liquid composition for applying a non-leachable antimicrobial layer or coating on a surface, comprising a solution, dispersion or suspension of a biguanide polymer, a cross-linker reacted with the biguanide polymer to form an adduct, and an antimicrobial metal, metal salt (e.g. a silver salt) or metal complex, wherein the metal, metal salt or metal complex forms a complex with the adduct, and wherein the antimicrobial layer or coating does not release biocidal levels of leachables into a contacting solution.

U.S. Pat. No. 5,817,325 relates to an article of manufacture having disposed on a surface thereof a contact-killing, non-leaching antimicrobial coating which kills microorganisms upon contact.

U.S. Pat. No. 5,849,311 further relates to a contact-killing, non-leaching antimicrobial material, capable of killing microorganisms which come into contact with the material.

U.S. Pat. No. 5,886,048 relates to a method of treating tumor disease in a human or animal with a therapeutically effective amount of PHMB or salt thereof.

U.S. Pat. No. 5,985,931 relates to an antimicrobial composition comprising defined amounts of PHMB, a quaternary ammonium compound; and parachlorometaxylenol wherein the combination of the three components is said to exhibit effective antimicrobial activity.

U.S. Pat. No. 5,990,174 relates to a method for improving haze formation and storage stability of an antimicrobial composition consisting of water and from 5 to 25% by weight of defined linear polymeric biguanide oligomers.

U.S. Pat. No. 5,993,840 relates to a composition comprising a non-woven material containing a mixture of polymeric biguanides subject to desorbtion when the non-woven material is wetted by urine; and an anionic polymer which is substantially insoluble in urine.

European Patent Application No. 136900 discloses the application of PHMB to a surgical drape comprising a non-woven fabric.

U.S. Pat. No. 6,017,561 discloses an antibacterial cleaning composition comprising a quaternary ammonium compound, an anionic polymer having an acid number greater than 10 wherein the anionic polymer is partially or completely neutralized by quaternary ammonium compound to form a polymer complex and wherein the polymer complex is greater than about 15% by weight of the solids in the composition, a dispersing agent, which comprises a surfactant that is selected from the group consisting of nonionic surfactant, amphoteric surfactant, and mixtures thereof, and optionally, a solvent. Also disclosed is a sponge device having antibacterial activity.

U.S. Pat. No. 6,160,196 relates to a wound covering comprising a hydrophobic, bacteria-absorbing synthetic or naturally-occurring polymer fiber material, having adhered thereto an antimicrobial active compound which is adapted to not be released into the wound.

U.S. Pat. No. 6,180,584 relates to a disinfectant composition comprising a film-forming antimicrobial material (which may be a polymeric biguanide material), an antimicrobial metallic material, and a carrier.

U.S. Pat. No. 6,235,302 relates to a sponge cloth which is based on regenerated cellulose and has been provided with an internal reinforcement consisting of, e.g., viscose fibers or cotton fibers having a staple fiber length of 5 to 50 mm.

### SUMMARY

An aspect of the present disclosure provides a medical dressing or wound dressing comprising an antimicrobial agent as well as a process of making such a medical dressing. It is a further aspect to provide a sponge formed from the medical dressing as well as a process of making such a sponge.

More particularly, the medical dressing and sponge formed therefrom provide an effective antimicrobial barrier for a wound while also controlling the release of antimicrobial agent contained within an inner portion of the dressing. The antimicrobial agent is preferably concentrated within an inner portion absorbent core of the dressing while an outer barrier layer portion reduces the release of the antimicrobial agent from the dressing.

A further aspect provide a medical dressing and a sponge formed therefrom wherein the outer hydrophobic layer(s) provide(s) non-adherent surfaces to aid in the healing of wounds and which reduces or eliminates pain experienced by a patient during changes in the dressing or sponge.

An aspect pertains to a medical dressing comprising layered fabric comprising an inner layer of substantially hydrophilic material; an outer layer of substantially hydrophobic material on both sides of the inner layer; and an antimicrobial agent, wherein the antimicrobial agent is contained in the inner layer.

In accordance with another aspect, the medical dressing may be in the form of a sponge comprising plies of the medical dressing material.

One or more further aspects pertains to a wound dressing which is constructed to accelerate the wound-healing process. According to an additional optional aspect, the wound dressing can be constructed such that it retains its wound-healing properties for an extended period of time, and thus does not have to be changed as frequently as conventionally-constructed wound dressings. According to another aspect, the wound dressing can possess increased effectiveness in preventing infection.

In some cases, the wound dressing can be provided with a combination of additives which, when provided in a wound dressing according to the teachings contained herein serve to increase the effectiveness of the wound dressing to promote healing relative to conventionally-constructed wound dressing materials containing conventional antimicrobial agents. In further cases, the wound dressing can be provided which generally contains a higher degree of antimicrobial agent, such as PHMB, than is typically contained in comparable wound dressings. Through specific wound dressing constructions and targeted and/or controlled release of antimicrobial and other agents, the wound dressing can increase its effectiveness over an extended period of time relative to conventional wound dressing constructions and compositions.

Consistent with the above, according to one optional aspect, increased control of bioburden is provided, without necessarily resorting to increased concentrations of antimicrobial agents, such as PHMB. According to a further aspect, the wound dressing is provided which reduces the risk of infection, or facilitates the control of an existing infection, without change to the existing wound care protocol. According to yet a further optional aspect, there is provided a wound dressing which will effectively increase the spectrum of activity of the antimicrobial agent contained therein. According to another optional aspect, a wound dressing is provided which provides targeted and/or controlled delivery of an antimicrobial agent and/or additional additives contained in the wound dressing to the wound site. According to yet another optional aspect, the dressing can promote migration of microbes from the wound bed into the dressing where they are then killed, and/or prevent migration of microbes from the external environment through the dressing so that they are killed before reaching the wound site.

According to one aspect, a multi-layer wound dressing can comprise at least one interior layer, the at least one interior layer containing PHMB or a PHMB derivative in an amount of at least about 3,000 ppm; and at least a first outer layer, the first outer layer containing PHMB or a PHMB derivative in an amount less than the amount of PHMB or PHMB derivative contained in the at least one interior layer.

The wound dressing can alternatively comprise a multi-layer structure comprising at least one interior layer, the at least one interior layer containing PHMB or a PHMB derivative in an amount of at least about 30,000 ppm; a first outer layer, the first outer layer containing PHMB or a PHMB derivative in an amount of at least 10,000 ppm; and a second outer layer, the second outer layer containing PHMB or a PHMB derivative in an amount of at least 10,000 ppm.

The multi-layer wound dressing can comprise at least one interior layer; and at least one exterior layer; wherein at least one of the interior and exterior layers contains PHMB or a PHMB derivative, and the other layer comprises a chelating agent.

The wound dressing can comprise at least one interior layer; and at least one exterior layer; wherein at least one of the interior and exterior layers contains PHMB or a PHMB derivative, zinc or a zinc-containing agent, or both.

The multi-layer wound dressing can comprise at least one interior layer comprising a woven, non-woven, foam, gel, film, or a mixture thereof, the at least one interior layer containing at least one of PHMB or a PHMB derivative and zinc or a zinc-containing compound; and at least one exterior layer comprising calcium alginate, PHMB or a PHMB derivative, and a zinc-containing agent.

The multi-layer wound dressing can comprise at least one interior layer containing an antimicrobial agent; and at least one exterior layer containing a cell-signaling agent.

A wound dressing formed according to yet another alternative configuration can comprise a wound dressing comprising a layer of cellulose or cellulose-based material containing at least about 10,000 ppm of PHMB or PHMB derivative.

One or more aspects can be directed to a multi-layer wound dressing comprising at least one interior layer of hydrophilic material having polyhexamethylene biguanide (PHMB) or a PHMB derivative in an amount of at least about 5,000 ppm; and a first outer layer of hydrophobic material adjacent the at least one interior layer.

One or more aspects can be directed to a method of preparing a medical dressing comprising providing a layered fabric consisting essentially of an inner layer of hydrophilic material, and outer layers of hydrophobic material on both sides of the inner layer; and impregnating a biguanide antimicrobial agent into the inner layer.

“Containing” or “contains” is to be broadly construed to mean that the one or more layers themselves and/or the materials making up the layers are impregnated with, and/or have coatings/treatments of other materials/agents applied thereto. The materials/agents may be applied to all or a portion of the layers or materials forming the layers. Finally, the term encompasses all methods or techniques of impregnation and/or coating/treatment, regardless of the state of the materials/agents being applied thereto (e.g., solid, liquid, gas, plasma, etc.). The added materials/agents can be applied during manufacture, or subsequent thereto (e.g., by the user/consumer prior to application of the one or more layers to the wound site). The terms also do not preclude the presence of other substances or materials, and should be construed as being equivalent to the term “comprising” in this regard.

As used herein, “PHMB” refers to polyhexamethylene biguanide, and “PHMB derivative” refers to polymeric biguanides that are cationic, displace divalent cations from the wall and membrane of bacteria and bring about disruption of the lipid bilayer. PHMB derivatives include, but are not limited to polyethylene hexamethylene biguanide (PEHMB) chlorohexadine glucomate, biodegradable PHMB, and other members of the biguanide family of antimicrobials.

As used herein, “interior layer” refers to a location within the dressing that is not intended to be directly applied to the surface of the skin or to a wound bed. As used herein, “exterior layer” refers to a location that (i) has a surface adapted to contact the surface of the skin or the wound bed and an opposing surface in contact with an interior layer, or (ii) a surface that faces away from the surface of the skin or wound bed and is exposed to the external environment, as well as an opposing surface for contact with an interior layer or surface of the dressing.

As used herein, “parts per million” or “ppm” refers to the amount of agent or substance contained within the dressing as determined by extracting the agent or substance out of the dressing material, and measuring the weight of extracted material versus the dry weight of the dressing material. For example, extraction can be conducted by soaking the dressing loaded with the agent or substance in 0.9% NaCl in water by weight (Isotonic saline) or 1M acetic acid overnight at a temperature of approximately 56.degree. C. The agent or substance in the resulting solution was identified via UV spectrophotometer or HPLC. This value is quantified by plotting the resulting peak against the standard dilution curve. The resulting loading level of agent or substance can then be calculated on a “ppm” basis.

As used herein “microbially-derived” or “microbially-derived” refers to cellulose or cellulose-based material formed consistent with the teachings of U.S. Patent Application Publication Nos. 2004/0028722, 2004/0142019, and 2005/0019380, and which is distinguished from plant-derived cellulose.

### BRIEF DESCRIPTION OF THE FIGURES

FIG. 1 is a schematic illustration of an exemplary embodiment of a wound dressing.

FIG. 2 is a schematic cross-sectional illustration, taken along lines 2-2 of FIG. 1 and can represent alternative embodiments of a wound dressing.

FIG. 3 illustrates a cross-sectional view of one form of the medical dressing containing two outer layers and an inner layer.

FIG. 4 illustrates a surface view of one form of the medical dressing containing a cross-shaped incision passing through all plies of the dressing.

### DETAILED DESCRIPTION

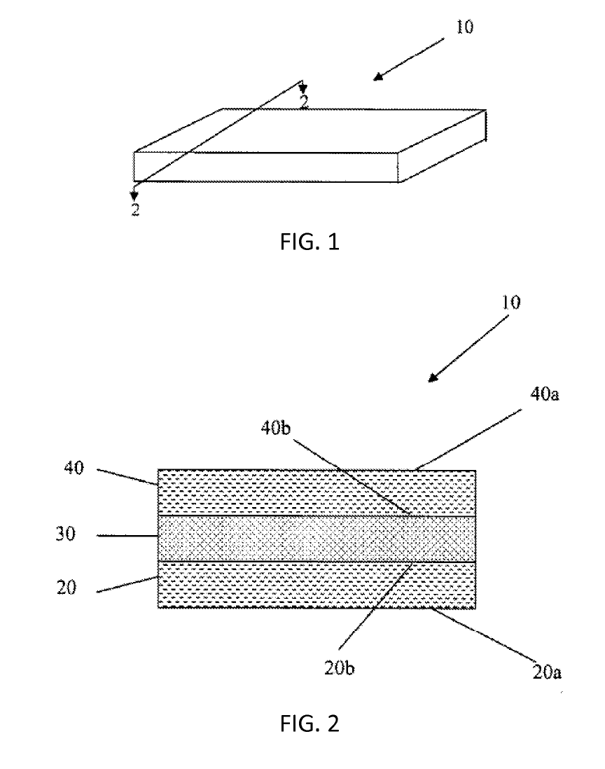

FIGS. 1-2 may be referred to in order to facilitate the following discussion. A wound dressing 10 can be generally formed from one or more discrete layers (e.g., 20, 30, 40). When composed of multiple layers, the dressing 10 includes at least one interior layer 30 as well as one or more exterior layer(s) 20, 40. The exterior layer(s) being characterized by at least one surface 20a adapted for contact with the surface of the skin of a wearer, or a wound bed, and an opposing surface 20b contacting an interior layer, or at least one surface 40a facing away from the surface of the skin or wound bed and exposed to the surrounding environment as well as an opposing surface 40b contacting an interior layer. The interior layer(s) 30 lack either a surface for contact with the skin surface or wound bed, or which is both exposed to the external environment and in contact with an interior layer.

While the illustrated embodiment includes one interior layer 30 and two exterior layers 20, 40, it should be understood that such embodiments are not limited to such construction. Any suitable number of layers may be present. According to certain exemplary embodiments, the dressing 10 can be in the form of a single layer. Alternatively, the dressing 10 can have only two layers. According to further alternative constructions, the dressing 10 can have more than three layers. For example, the dressing can have 4, 5, 6, 7, 8 or more layers.

The antimicrobial agent(s) and/or other components or agents identified herein can be added to the various interior and/or exterior layers of the dressing in any suitable manner. For example, the agent(s) can be sprayed onto the dressing layer(s), or the dressing layer(s) can be soaked or dipped in a solution containing the agent(s), then dried. The agent(s) can optionally be combined with the various interior and/or exterior layers of the dressing so as to render them releasable therefrom (e.g., so as to migrate out of the layer(s), toward, and into the wound bed). For example, the dressing can be moistened with a predetermined amount of isotonic saline (e.g., 0.9% Na) or sodium citrate, then combined with an antimicrobial, which can be contained in the saline or citrate medium, or added sequentially thereto.

In addition, due to the optional absorbent characteristics of the dressing, microbes are absorbed within the layer(s) of the dressing and killed by the antimicrobial and/or other agent(s) contained therein and prevented from passing through the dressing. Thus, it can be advantageous to combine the agent(s) with the dressing material in a manner that prevents substantial amounts of agent(s) from leaving the dressing. For example, the dressing can be cured at a specific pH level (e.g., pH=7+/−0.4). The agent(s) will only be released in large amounts when the pH of the dressing reduces around 5 or less, which is not a typical pH associated with wound exudate.

The interior layer(s) 30 may be substantially hydrophilic, while one or more of the outer layers 20,40 may be substantially hydrophobic. The term “substantially hydrophilic” describes the function of the inner layer material. It also distinguishes the inner layer material over the function of the “substantially hydrophobic” outer layer material, which can act to provide an antimicrobial barrier property and attenuates or reduces the release of antimicrobial agent from the interior layer(s) 30 away from the dressing. Retention of antimicrobial agent within the inner layer also lowers the bioburden, i.e., the growth and number of cells, within the dressing during use. The various layers of the dressing material can be provided with the desired hydrophilic or hydrophobic properties in accordance with any suitable known manner. The terms “substantially hydrophilic” and “substantially hydrophobic”, as they apply to the inner and outer layers respectively, may be related to the percentage moisture regain for fibers of the respective materials, typically at 70° F. and 65% relative humidity. The fibers used in the substantially hydrophobic outer layer should have a moisture regain of less than about 10%, preferably less than about 5%, and most preferably less than about 2% based on the weight of the dry fiber. The fibers used in the substantially hydrophillic inner layer should have a moisture regain of about 5-25, preferably about 10-20, and most preferably about 15-20 times the weight of the dry fiber.

Each of the one or more layers can be formed from any suitable material and/or construction. For example, the one or more layers can be formed from a material that is fibrous, film-like, gel, or combinations thereof. With respect to fibrous materials, they can be woven or nonwoven materials. The fibers can be selected from natural fibers, synthetic fibers, and combinations of the two. By way of non-limiting example, suitable materials which can be utilized to form the one or more layers may include: cellulose, non-microbially derived cellulose, cellulose acetate, oxycellulose, alginates, cotton, polypropylene, polyvinyl alcohol, rayon, nylon, acrylic, polyester, polyurethane, hydrogels, hydrocolloids and combinations thereof.

According to one optional embodiment, at least one exterior layer 20, 40 can be constructed to be dissolvable or absorbable. Accordingly, one or more layers of the dressing may comprise a bioabsorbable material such as polyglycolic acid, polylactic acid, collagen, chitin, keratin, an alginate, guar gum, locust bean gum or derivatives or mixtures thereof. The layer also may comprise a bioabsorbable polymer formed by chemically modifying a natural substance, for example, oxidized cellulose or chitosan or a cross-linked hyaluronic acid gel.

A number of alternative antimicrobial agents may be utilized. Suitable antimicrobial agents include, but are not limited to, a chlorohexidine, a chlorohexadine salt, a triclosan, a polymoxin, a tetracycline, an amino glycoside (e.g., gentamicin or TOBRAMYCIN™), a rifampicin, a bacitracin, an erythromycin, a neomycin, a chloramphenicol, a miconazole, a quinolone, a penicillin, a nonoxynol 9, a fusidic acid, a cephalosporin, a mupirocin, a metronidazole, a secropin, a protegrin, a bacteriolcin, a defensin, a nitrofurazone, a mafenide, a acyclovir, a vanocmycin, a clindamycin, a lincomycin, a sulfonamide, a norfloxacin, a pefloxacin, a nalidizic acid, an oxalic acid, an enoxacin acid, a ciprofloxacin, a biguanide (e.g., PHMB), combinations thereof and the like. In certain embodiments the antimicrobial agent can comprise polyhexamethylene biguanide (PHMB) or a derivative thereof. The antimicrobial agent can be present in the dressing at any suitable level which provides an adequate an adequate antimicrobial effect. For example, suitable concentration levels include, but are not limited to, 2,000 ppm, 2,500 ppm, 3,000 ppm, 3,500 ppm, 5,000 ppm, 10,000 ppm, 13,000 ppm, 30,000 ppm, and combinations and/or gradients thereof. According to one optional embodiment, the dressing contains PHMB or a PHMB derivative present in any of the above-listed amounts. In general, the antimicrobial agent may be any such agent which suitably functions to provide an antimicrobial property to the inventive medical dressing.

The wound dressing may further include a chelating agent, as an additional component or as a full or partial substitute for any of the above. Any suitable chelating agent may be utilized. By way of non-limiting example, chelating agents such as ethylenediaminetetraacetic acid (EDTA), variations of EDTA such as, for example, disodium EDTA or tetrasodium EDTA, combinations thereof and the like, are contemplated. Other chelating agents such as citrate and heprin are also contemplated. Chelating agents can heighten the susceptibility of bacteria and other organisms to the antiseptic effects of another antimicrobial agent, thereby rendering the wound dressing more effective in combating and/or preventing infection. Generally, chelating agents advantageously (i) are non-thrombogenic; (ii) are more active in an acidic environment; (iii) re-sensitize microbes to the effects of other antimicrobial agents; (iv) provide a debriding effect and (v) remove ionic attractions necessary to form/sustain biofilms. This aspect can advantageously avoid or at least reduce the likelihood of problems caused by the potentially irritating effects of certain antimicrobial agents, such as PHMB, especially when applied to the skin at higher concentration levels. The chelating agent can be present at any suitable concentration. For example, the chelating agent can be present in amounts on the order of about 0.05 to about 1.0% by weight.

As an additional component, or as a full or partial substitute for one or more of the above-mentioned antimicrobial agents and/or chelating agents, the wound dressing can include one or more additional antimicrobial agents. By way of non-limiting example, suitable additional antimicrobial agents include, but are not limited to: polyethylene hexamethylene biguanide (PEHMB), silver, copper, and combinations thereof. The one or more additional antimicrobial agents can be present at any suitable concentration level. For example, the dressing can contain 1 to 3% by weight silver of the additional antimicrobial agent(s).

As an additional component, or as a full or partial substitute for one or more of the above-mentioned antimicrobial agents, chelating agents and/or additional antimicrobial agents, the dressing may further include a zinc-containing agent. Suitable zinc-containing agents include, but are not limited to, zinc, zinc alginate, zinc bacitracin, zinc oxide, zinc phosphate, zinc aspartate, and combinations thereof. According to one optional embodiment, the zinc-containing agent includes zinc acetate, zinc butyrate, zinc citrate, zinc gluconate, zinc glycerate, zinc glycolate, zinc formate, zinc lactate, zinc picolinate, zinc propionate, zinc salicylate, zinc tartrate, zinc undecylenate, Zinc Stearate and combinations thereof. Zinc-containing agents can improve the rate of wound healing, thereby rendering the wound dressing more effective in combating and/or preventing infection, without the necessity of increasing the levels of antimicrobial agent contained therein. Combination with an aliginate provides moisture-absorption capabilities, and alginates help promote a moist wound healing environment. This aspect can advantageously avoid or at least reduce the likelihood of problems caused by the irritating effects of certain antimicrobial agents, such as PHMB, especially when applied to the skin that higher concentration levels.

As an additional component, or as a full or partial substitute for one or more of the above-mentioned antimicrobial agents, chelating agents, additional antimicrobial agents, and/or zinc-containing agents, the dressing may further include a cell-signaling agent. A cell-signaling agent provides a mechanism for communicating with the cell by electrical, chemical or biologic means that encourages cell growth or movement or receptive action in the direction of the signal. The signal may also deactivate the bacterial cells' defense mechanisms. According to this construction, bacterial growth is promoted in a preferred manner (i.e., away from the wound bed) which leads to an increased efficacy of the wound dressing.

Exemplary wound dressings can include additional active ingredients or agents such as, for example, a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to induce directional bacterial growth, an agent to impart bactericidal or bacteriostatic activity, an electron transfer agent to destabilize or destroy the metabolic action of microbes and/or biofilm formation, combinations thereof and the like. These agents can be present in the dressing in any suitable amount, such as about 0.05 to about 1.0% by weight. Release of active agents may be triggered by a variety of means, such as, for example, an electric field or signal, temperature, time, pressure, moisture, light (e.g., ultra-violet light), ultrasound energy, sonication, combinations thereof and the like. By way of non-limiting example, the additional agent can comprise silver or compounds thereof.

Any of the above-mentioned components or agents may be combined directly with the material forming the one or more layers of the wound dressing in any conventional manner. Alternatively, any of the above-mentioned agents may be contained, and subsequently released, by a delivery agent. Any suitable delivery agent can be utilized. By way of non-limiting example, suitable delivery agents include: a hydrogel, phosphate glass, powdered carrier, or a film carrier.

The antimicrobial, chelating agent, additional antimicrobial agent, zinc-containing agent, cell-signalling agent and/or or other agent mentioned above can optionally be printed or otherwise applied to one or more layers of a wound dressing to provide a desired concentration or concentration gradient of one or more of these agents on and/or within the dressing. For example, one or more of the agents can be applied separately, or in combination, in a specific pattern corresponding to the wound area, for the purpose of optimizing the antimicrobial and wound healing effects.

The wound dressing can comprise one or more layers containing at least one antimicrobial agent and at least one chelating agent. According to one optional configuration, all layers of the wound dressing may contain a combination of antimicrobial agent and chelating agent. The antimicrobial agent can be present in amounts of about 2500 to about 30,000 ppm. The chelating agent can be present in amounts of about 1,000 to about 10,000 ppm.

According to another alternative modification of the above multi-layer configuration, the antimicrobial agent and the chelating agent can be separately contained in different layers of the wound dressing. Thus, for example, a wound dressing can be formed with at least one interior layer (e.g., 30), and at least one exterior layer (e.g., 20, 40). The antimicrobial agent can be contained in the interior layer 30, which is not in direct contact with the skin or wound, and the chelating agent can be provided in one or more exterior layer(s) 20, 40. According to one embodiment, the interior layer 30 contains about 30,000 ppm PHMB or a derivative thereof and one or more exterior layers 20, 40 contain about 5,000 ppm EDTA. According to an further optional embodiment, the interior layer(s) 30 can be constructed so as to prevent the escape of a significant amount of antimicrobial agent therefrom, while at least one of the exterior layers can be constructed so as to permit the migration of the chelating agent into the surface of the skin or wound bed. As an optional modification of the above, the chelating agent can be provided in the interior layer 30, and the antimicrobial agent provided in one or more of the exterior layers 20, 40.

According to a further alternative construction, the wound dressing 10 is formed from a plurality of different layers and materials containing agents to enhance performance. A cell-signaling agent of the type described above can be provided in the dressing between the wound bed and another dressing layer which is treated with one or more of the antimicrobial agents identified herein. For example, an exterior layer 20 can be provided which contains the cell-signaling agent, and an interior layer 30 can be provided that contains one or more antimicrobial agent(s) including PHMB or a derivative thereof. According to this construction, bacteria would need to cross the antimicrobial agent to reach the signaling mechanism, and bacterial growth is promoted in a preferred manner (i.e., away from the wound bed) which leads to an increased efficacy of the wound dressing.

In an alternative configuration, the wound dressing 10 can comprise one or more layers containing at least one antimicrobial agent and/or at least one zinc-containing agent. According to one optional modification, all layers of the wound dressing may contain a combination of the antimicrobial agent and zinc-containing agent.

According to another alternative modification of the above configuration, the wound dressing comprises a plurality of layers and the antimicrobial agent and the zinc-containing agent can be separately contained in different layers of the wound dressing. As one possible example of this configuration, at least one of the layers contains both an antimicrobial agent and a zinc-containing agent, while other layer(s) separately contain the antimicrobial agent or zinc-containing agent.

For example, the interior layer(s) 30, which is not in direct contact with the skin or wound, contains PHMB or a derivative thereof, and the zinc-containing agent can be provided in one or more of the outer layers 20, 40.

According to an alternative construction, at least one interior layer 30 and at least one exterior layer 20, 40 each contain a combination of PHMB or a derivative thereof and zinc-containing agent. The layers may have different concentrations of the PHMB or derivative thereof and/or zinc-containing agent. According to one embodiment, layer 30 contains about 30,000 ppm PHMB, and layers 20, 40 each contain about 0.1 to about 3.0% zinc alginate by weight.

According to a further alternative construction, at least one interior layer 30 contains a combination of PHMB or a derivative thereof and zinc, and at least one exterior layer 20, 40 can contain a zinc-containing agent.

According to yet another alternative configuration, at least one interior layer 30 contains a combination of PHMB or a derivative thereof and a zinc-containing agent, and at least one exterior layer 20, 40 contains a combination of calcium alginate, PHMB or a derivative thereof, and a zinc-containing agent.

The multi-layer dressings can have layers with different concentrations of antimicrobial agent.

According to one optional configuration, a dressing can be constructed such that it is provided with at least one interior layer and at least one exterior layer both which contain an antimicrobial agent in different amounts. Specifically, at least one interior layer contains a relatively higher concentration of antimicrobial agent and at least one of the exterior layers contained in the dressing.

According to one optional embodiment, the dressing contains at least one interior layer 30 which contains PHMB or a PHMB derivative in an amount of at least 3,000 ppm, and at least one exterior layer which contains PHMB or a PHMB derivative in an amount which is less than 3,000 ppm. According to various optional modifications of this construction, the at least one interior layer 30 can contain PHMB or a PHMB derivative in amounts of at least 3,500 ppm, at least 5,000 ppm, at least 10,000 ppm, at least 13,000 ppm, or at least 30,000 ppm, while the at least one exterior layer 20, 40 of the dressing also contains PHMB or a PHMB derivative in an amount which is less than the amount contained in the at least one interior layer 30.

According to one optional, and more specific embodiment of the above described construction, a dressing can be provided having at least one interior layer 30 which contains at least about 13,000 ppm of PHMB or a PHMB derivative, and at least one exterior layer 20, 40 which contains at least about 2,000 ppm PHMB or PHMB derivative. According to one variation of this embodiment, the dressing comprises two exterior layers 20, 40, each containing at least about 2,000 ppm PHMB or PHMB derivative.

According to another optional, and more specific embodiment of the above described construction, addressing can be provided having at least one interior layer 30 which contains at least about 30,000 ppm PHMB or PHMB derivative, and at least one exterior layer 20, 40 which contains at least about 10,000 ppm PHMB or PHMB derivative. According to one variation of this embodiment, the dressing comprises two exterior layers 20, 40, each containing at least about 10,000 ppm PHMB or PHMB derivative. According to one optional embodiment, the at least one interior layer 30 can be constructed to prevent elution of substantial amounts of PHMB or PHMB derivative into adjacent layers of the dressing and/or into the skin or wound bed. According to a further optional embodiment of the above, the at least one exterior layer 20, 40 can be constructed so as to permit PHMB or PHMB derivative to elute into the skin or wound bed.

The wound dressing can comprise a layer formed primarily from a cellulose or cellulose-based material which contains PHMB or a PHMB derivative. The cellulose or cellulose-based dressing material can comprise at least about 50% cellulose material. According to one optional embodiment the dressing material comprises 100% cellulose material. The cellulose material may optionally comprise rayon. According to one optional embodiment, dressing can include a layer of cellulose or cellulose-based material which contains at least 5000 ppm PHMB or PHMB derivative. According to certain optional modifications of this construction, a layer of cellulose or cellulose-based material can contain PHMB or PHMB derivative in amounts of at least about 10,000 ppm, at least about 13,000 ppm, or at least about 30,000 ppm. According to a further optional modification of this embodiment, the cellulose or cellulose-based material is not microbially-derived. According to yet another optional modification of this construction, the dressing can be formed of a single layer, without any additional layers contained therein.

In one embodiment, the inventive medical dressing is based in part upon the dressing disclosed in U.S. Pat. No. 4,211,227, the disclosure of which is incorporated herein by reference in its entirety.

In one embodiment, the inventive medical dressing is an antimicrobial drain sponge which is a thermally bonded nonwoven absorbent material, typically in the form of a square that is 2″×2″ or 4″×4″ and having a 6 ply thickness. The material is preferably about 50% polyester and about 50% rayon with each ply formed as a sandwich of a layer of rayon between two layers of polyester.

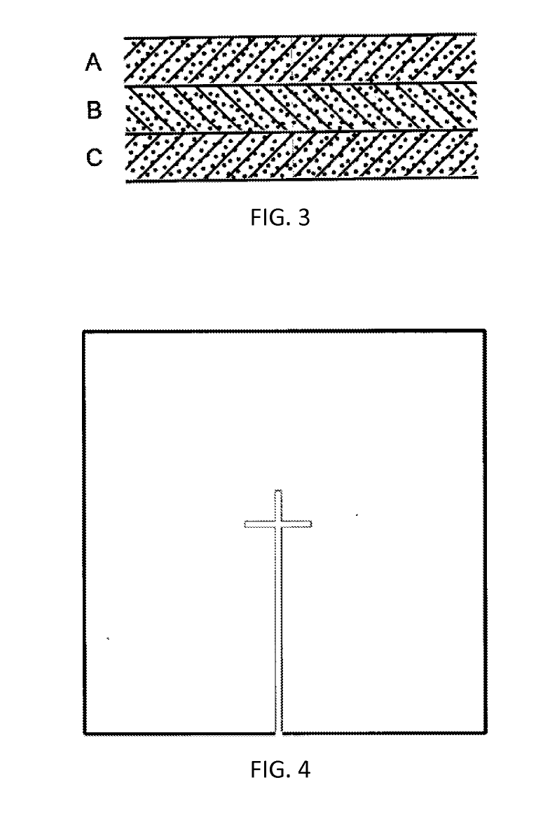

As illustrated in FIG. 3, the wound dressing can have outer layers (A and C) formed from a hydrophobic material, preferably hydrophobic fibers, such as polyester fibers. The inner layer (shown as layer B in FIG. 3) can comprise a hydrophilic material, preferably hydrophilic fibers, such as cellulosic fibers. More preferably, the cellulosic fibers are rayon.

In order to impart antimicrobial properties, the material that is to be cut into the individual sponges is impregnated with an aqueous solution of an antimicrobial agent, such as polyhexamethylene biguanide (PHMB). The PHMB is suitably present in a concentration of about 0.1 to 0.5% by weight, preferably about 0.15 to 0.35% by weight, and most preferably about 0.2 to 0.3% by weight. A surfactant may also be present in the aqueous solution. For example, commercially available non-ionic surfactants, such as TWEEN 20, which is polyoxyethylene (20) sorbitan monolaurate, may be utilized. PHMB used to prepare the drain sponge may be provided by, e.g., Avecia Inc. under the designation COSMOCIL CQ (an aqueous solution of PHMB containing 19-21% w/w active ingredient and a pH of 5.0-5.5). The material may be impregnated with the antimicrobial agent according to any suitable technique, such as, e.g., by dip coating the material in a solution comprising the antimicrobial agent.

The impregnated material may then be pressed at a pressure designed so that the appropriate level of antimicrobial agent, preferably PHMB, remains in the material. After drying by passing between heated rollers, the material has a residual antimicrobial agent (e.g. PHMB) content of from 1500 to 3500 ppm. The material is subsequently converted into 4″×4 and 2″×2″, 6 ply drain sponges. The sponges do not include an adhesive, but have a cross-shaped incision in the center (as illustrated in FIG. 4) that penetrates all 6 plies so that a drain tube can be passed therethrough. The antimicrobial agent (e.g. PHMB) can preferably be released from the medical dressing or sponge in a moist environment to provide antimicrobial activity beyond the edges of the sponge for a limited time. In general, the fabric is treated to have about 1500-3500 ppm of extractable antimicrobial agent, preferably polyhexamethylene biguanide.

The antimicrobial agent is preferably releasably impregnated into the inner layer substantially hydrophilic material of the fabric, coated on said inner layer or a combination thereof. The antimicrobial agent is also preferably controllably releaseable from the fabric in an amount effective as an antimicrobial, more preferably in amount effective as an antimicrobial for a period of up to about 2 days, most preferably up to about 1 day. The antimicrobial amount is defined as the amount of antimicrobial agent applied to the dressing that is needed for providing sufficient antimicrobial characteristics, yet without causing irritation of the skin or open wound.

Although the antimicrobial agent may be released from the inner layer material of the fabric, the antimicrobial treatment of the fabric principally allows the dressing to function as a barrier to contamination of the wound from sources outside the wound. In addition, due to the absorbent characteristics of the dressing, microbes absorbed within the inner layer are prevented from escaping through the dressing.

The fabric inner layer material is substantially hydrophilic, and is preferably substantially a cellulose fiber, and, more preferably, is substantially rayon. Other inner layer materials may also be utilized, including polysaccharides, alginates, cotton or carboxy methyl cellulose fiber materials.

Hydrophobicity and hydrophilicity are commonly reported as the percentage moisture regain for fibers of the material.

The fabric outer layer material is preferably substantially polyester, and, more preferably, is a combination of textile matrix grade polyester fiber and amorphous binder grade polyester fiber. The binder fibers function to bind, e.g., rayon and textile grade polyester fibers together. Suitable inner layer materials include, e.g., Lenzing 8191 rayon. Suitable outer layer materials include, e.g. Wellman Polyester T-203 textile grade polyester and Kosa Polyester 259 amorphous binder grade polyester. The textile to binder fiber weight ratio is preferably about 2:1. Other materials may also be utilized for the outer layer, including, e.g., polyolefins such as polyethylene or polypropylene, nylons, or other thermoplastic fibers.

The hydrophobic layers of the fabric forming the medical dressing may also be imparted with a hydrophilic finish. Such a finish may be applied as a coating of a hydrophilic finishing agent on the outer layers. It may also be applied during the impregnation of the inner layer with the antimicrobial agent by including the hydrophilic finishing agent in the same solution of antimicrobial agent. By contacting the dressing material with the antimicrobial solution containing a hydrophilic finishing agent, the inner layer is impregnated with the antimicrobial solution while the outer layers are imparted with a hydrophilic finish. As used herein, the term “hydrophilic finish” is intended to mean that the hydrophilic characteristics of the outer layer are increased at least to some degree by the use of a hydrophilic finishing agent. In a preferred embodiment, the hydrophilic finish helps to improve the function of the dressing material or sponge by allowing wound exudate to be more easily absorbed by the inner layer absorbent core material. Suitable hydrophilic finishing agents include, e.g., surfactants, such as non-ionic surfactants, which may be typically applied in concentrations of about 0.1% by weight. As noted above, one such non-ionic surfactant is TWEEN 20, which is polyoxyethylene (20) sorbitan monolaurate, although other such surfactants may also be utilized.

A medical sponge formed from plies of the medical dressing material may contain several plies of the dressing material, preferably 2-10 plies, more preferably 4-8 plies, and most preferably 6 plies. A cross-shaped incision may be preferably formed in the center of the sponge that penetrates all plies, thereby allowing an object to pass through the sponge.

In a preferred aspect, the fabric comprises about 50% by weight rayon, about 33% by weight textile matrix grade polyester, and about 17% by weight binder grade polyester, based on the weight of the fabric.

The fabric of the medical dressing and sponge may be processed to impart softness and improved absorbency, according to techniques known in the art, followed by further processing to form the inventive medical dressing and sponge. Suitable techniques include the use of microcreping devices such as are commercially available from Micrex, Corp. for the treatment of nonwoven and other materials. It is preferred that such treatments provide an absorbency of from about 6-15, preferably 10-15, times the dry weight of the fabric.

The term “absorbency” refers to the amount of liquid material, such as water or wound exudate, that may be absorbed by the fabric of the medical dressing or sponge.

Compared with other commercial medical dressings and sponges, the medical dressings and sponges provides several advantages and benefits. For example, by containing the antimicrobial agent within the inner layer of the medical dressing and the sponge formed therefrom, the release of the antimicrobial agent is controlled such that sensitive wound or skin areas are not adversely irritated. The presence of the antimicrobial agent within the inner layer also allows for the dressing and sponge to function effectively as a barrier against contamination of the wound. Contamination of areas outside the wound and dressing or sponge due to the retention of the wound exudate within the dressing or sponge is also reduced or prevented. In addition, the use of the outer hydrophobic (nonwoven) layers provides the dressing and sponge with the characteristic of non-adherency to wounds. Further, due in part to the presence of the outer hydrophobic layer(s), the amount of antimicrobial agent within the inner layer necessary to maintain the antimicrobial effectiveness of the dressing or sponge is also reduced compared with other commercial products.

The medical dressing may also comprise layered fabric consisting essentially of an inner layer of substantially hydrophilic material; an outer layer of substantially hydrophobic material on both sides of the inner layer; and an antimicrobial agent, wherein the antimicrobial agent is contained in the inner layer.

As used in the context herein, the phrase “consisting essentially of” is intended to mean that the certain additional components which would materially affect the basic and novel characteristics of the inventive medical dressing are not included in the layered fabric. As concerns the medical dressing and sponge, “consisting essentially of” is intended to mean, e.g., that adhesives are not utilized to prepare the layered fabric or that additional agents are not utilized to bind the antimicrobial agent within the inner layer material of the medical dressing.

The entirety of each of U.S. Pat. No. 8,100,872, titled MEDICAL DRESSING CONTAINING ANTIMICROBIAL AGENT, U.S. Patent Application Publication Nos. 2012/0089069, titled MEDICAL DRESSING CONTAINING ANTIMICROBIAL AGENT, 2007/0237812, titled MULTI-LAYER WOUND DRESSINGS, and 2012/0177720, titled MULTI-LAYER WOUND DRESSINGS, and U.S. Patent Application Nos. 60/790,813 and 60/790,814, is incorporated herein by reference for all purposes.

### EXAMPLES

The antimicrobial efficacy of the medical dressing and sponge has been investigated. Such tests simulate the use of the dressing or sponge in practice and are based upon the visual observation of microbial activity on growth media placed in contact with the inventive medical dressing or sponge containing a determined amount of microbes. The tests are generally conducted by placing a sample of the antimicrobial agent containing dressing or sponge in contact with growth medium, adding a small volume of microbe containing aqueous solution to the medical dressing or sponge, and incubating the test samples for definite time periods and under controlled temperature and environmental conditions. Following the end of the pre-determined time period, the growth medium is visually observed to determine the extent, if any, of microbial activity or growth on the medium. An assessment of the antimicrobial effectiveness of the inventive dressing and sponge against various microbes, including S. aureus, S. epidermidis, E. coli, and P. aeruginosa, has been conducted according to this procedure. In all such tests, the medical dressing and sponge has been determined to be effective to prevent microbial activity on such growth medium. By comparison, control samples not containing an antimicrobial agent, such as PHMB, were determined using the same procedures and conditions to not prevent microbial activity on such growth medium.

While the disclosure has been described in detail by reference to specific embodiments, the skilled artisan will appreciate that various modifications, substitutions, omissions and changes may be made, and equivalents employed, without departing from the spirit of the invention or the scope of the appended claims.

1. A multi-layer wound dressing comprising:
at least one interior layer of hydrophilic material having polyhexamethylene biguanide (PHMB) or a PHMB derivative in an amount of at least about 5,000 ppm; and
a first outer layer of hydrophobic material adjacent the at least one interior layer.

2. The dressing of claim 1, wherein the at least one interior layer is constructed to prevent elution of PHMB or PHMB derivatives into adjacent layers of the wound dressing and/or into the skin or wound bed.

3. The dressing of claim 1, wherein the PHMB or PHMB derivative is releasably contained in the interior layer.

4. The dressing of claim 1, wherein the at least one interior layer has at least about 10,000 ppm of PHMB or PHMB derivative.

5. The dressing of claim 1, further comprising a second outer layer of hydrophobic material.

6. The dressing of claim 6, wherein at least one of the first and second outer layers is dissolvable or absorbable.

7. The dressing according to claim 6, wherein the PHMB or PHMB derivative is coated on the at least one interior layer.

8. The dressing of claim 1, wherein the at least one interior layer has at least about 13,000 ppm of PHMB or PHMB derivative.

9. The dressing of claim 1, further comprising a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to induce directional bacterial growth, an agent to impart bactericidal or bacteriostatic activity, an electron transfer agent to destabilize or destroy the metabolic action of microbes or biofilm formation, and combinations thereof.

10. The dressing of claim 1, wherein one or more of the interior and the first outer layers is cotton, polypropylene, polyvinyl alcohol, polyester, rayon, polyurethane, acrylic, cellulose, cellulose acetate, alginate, or combinations thereof.

11. The dressing of claim 1, wherein the first outer layer of hydrophobic material has a zinc-containing agent.

12. The dressing of claim 11, wherein the at least one interior layer has at least 2,500 to about 30,000 ppm of PHMB or PHMB derivative and about 1.0-3.0% by weight of the zinc-containing agent; and the first outer layer has at least about 1,500 to about 3,500 ppm of PHMB or PHMB derivative and about 0.1 to 1.0% by weight of the zinc-containing agent.

13. The dressing of claim 11, wherein the zinc-containing agent is zinc, zinc alginate, zinc bacitracin, zinc oxide, zinc phosphate, or zinc aspartate.

14. The dressing of claim 11, wherein the at least one interior layer has at least about 13,000 ppm of PHMB or PHMB derivative, and the first outer layer has at least about 2,000 ppm of a cell-signaling agent.

15. A method of preparing a medical dressing comprising:
providing a layered fabric consisting essentially of an inner layer of hydrophilic material, and outer layers of hydrophobic material on both sides of the inner layer; and
impregnating a biguanide antimicrobial agent into the inner layer.

16. The method according to claim 15, wherein impregnating the biguanide antimicrobial agent comprises contacting the layered fabric with a solution having a polyhexamethylene biguanide.
